(19) United States
(12) Patent Application Publication
(10) Pub. No.: US 2025/0258107 A1
(43) Pub. Date: Aug. 14, 2025

(54) FOOD INSPECTION APPARATUS

(71) Applicant: Konica Minolta, Inc., Tokyo (JP)

(72) Inventors: Yuki ONO, Tokyo (JP); Junichi JONO, Tokyo (JP); Hiroki SHIBATA, Kobe-shi (JP); Makoto OKI, Tokyo (JP)

(21) Appl. No.: 19/044,147

(22) Filed: Feb. 3, 2025

(30) Foreign Application Priority Data
Feb. 14, 2024 (JP) 2024-020538

(51) Int. Cl. G01N 21/88; G01N 21/84; G01N 21/93; G01N 33/02

(52) U.S. Cl. CPC G01N 21/8851; G01N 21/93; G01N 2021/845; G01N 2021/8887; G01N 33/02; G01N 2201/0231

(57) ABSTRACT

A food inspection apparatus configured to inspect food being conveyed by a conveyance mechanism includes an optical unit and a transparent window member. The optical unit is disposed above the conveyance mechanism and includes an imager. The imager is configured to image the conveyed food and acquire inspection information on the food. The transparent window member is disposed between the conveyance mechanism and the optical unit.

### CROSS-REFERENCE TO RELATED APPLICATIONS

The entire disclosure of Japanese Patent Application No. 2024-020538 filed on Feb. 14, 2024, is incorporated herein by reference in its entirety.

### BACKGROUND OF THE INVENTION

### Technical Field

The present invention relates to a food inspection apparatus.

### Description of Related Art

In food processing, the water content of food conveyed on a belt conveyor is inspected for stable quality control of food. To inspect the water content of food, an analytical apparatus, such as a multispectral camera, can be used that calculates the two-dimensional distribution of the water content of food in an evaluation area on the belt.

Japanese Unexamined Patent Publication No. H 6-109719 (A) discloses a quality evaluation method for calculating the water content of food, such as cooked rice. The quality evaluation method uses a near-infrared spectroscopic analyzer to calculate the water content in the cooked rice by multiple regression analysis of the wavelength of light reflected by the cooked rice. The near-infrared spectroscopic analyzer is disposed near the cooked rice outlet of the continuous rice cooker.

In the known art, the steam, oil splash, or the like from food conveyed by the conveyance device may stain the camera of the optical unit constituting the food inspection apparatus. Such stains prevent accurate inspections of the water content of food.

To deal with the above-described issue, an object of the present invention is to provide a food inspection apparatus capable of preventing contamination of an imager of an optical unit.

### SUMMARY OF THE INVENTION

To achieve at least one of the abovementioned objects, according to an aspect of the present invention, there is provided a food inspection apparatus configured to inspect food being conveyed by a conveyance mechanism, the food inspection apparatus including: an optical unit that is disposed above the conveyance mechanism and that includes an imager, the imager being configured to image the conveyed food to acquire inspection information on the food; and a transparent window member disposed between the conveyance mechanism and the optical unit.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a perspective view of a schematic configuration of a food inspection apparatus after an optical unit is set on a conveyance mechanism according to the present embodiment as an example;

FIG. 2 is a perspective view of the schematic configuration of the food inspection apparatus before the optical unit is set on the conveyance mechanism according to the present embodiment as an example;

FIG. 3 is a side view of a schematic configuration of the food inspection apparatus after the optical unit is set on the conveyance mechanism according to the present embodiment as an example;

FIG. 4 is a front view of a schematic configuration of the food inspection apparatus after the optical unit is set on the conveyance mechanism according to the present embodiment as an example;

FIG. 5 is a diagram of the internal configuration of the optical unit according to the present embodiment as an example;

FIG. 6 is a diagram of the configuration of a stand according to the present embodiment;

FIG. 7 is a diagram for explaining the flow of obtaining reference information that is used in calibration of spectral data obtained by the imager according to the present embodiment; and

FIG. 8 is a diagram of the internal configuration of the optical unit of the food inspection apparatus according to a modification example.

### DETAILED DESCRIPTION

Hereinafter, a food inspection apparatus according to a preferred embodiment of the present disclosure will be described in detail with reference to the drawings. However, the scope of the invention is not limited to the disclosed embodiments.

### Configuration Example of Food Inspection Apparatus 1

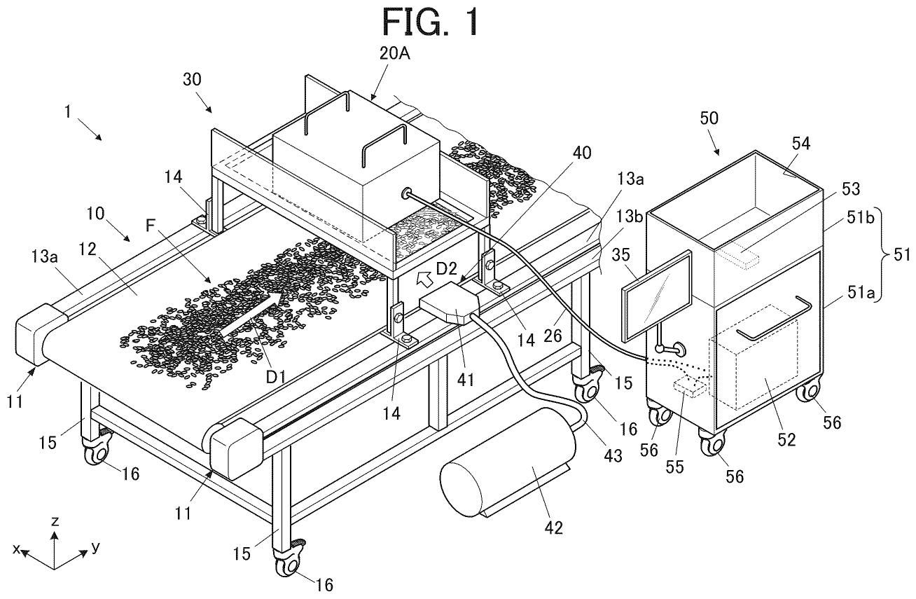

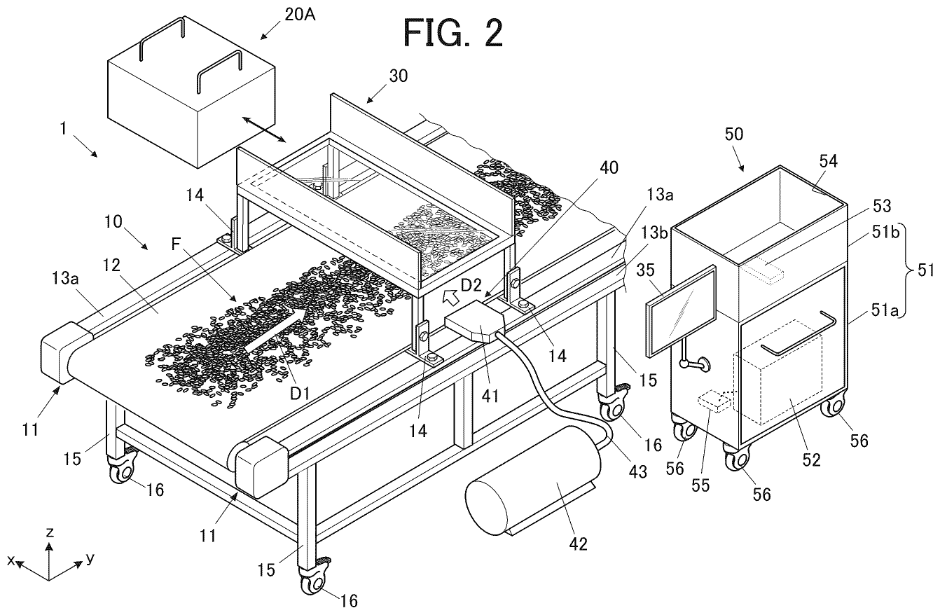

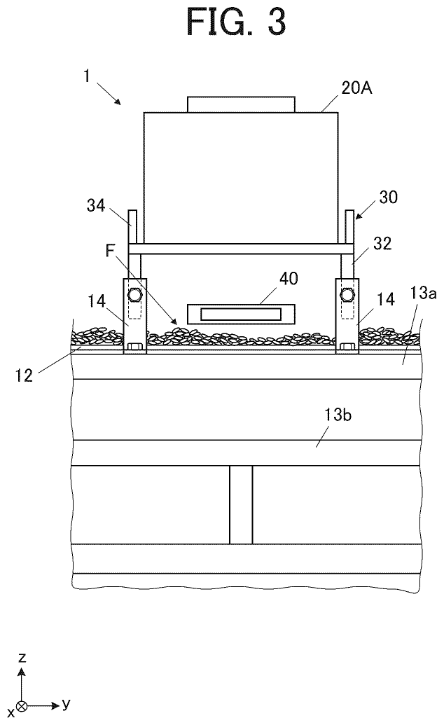

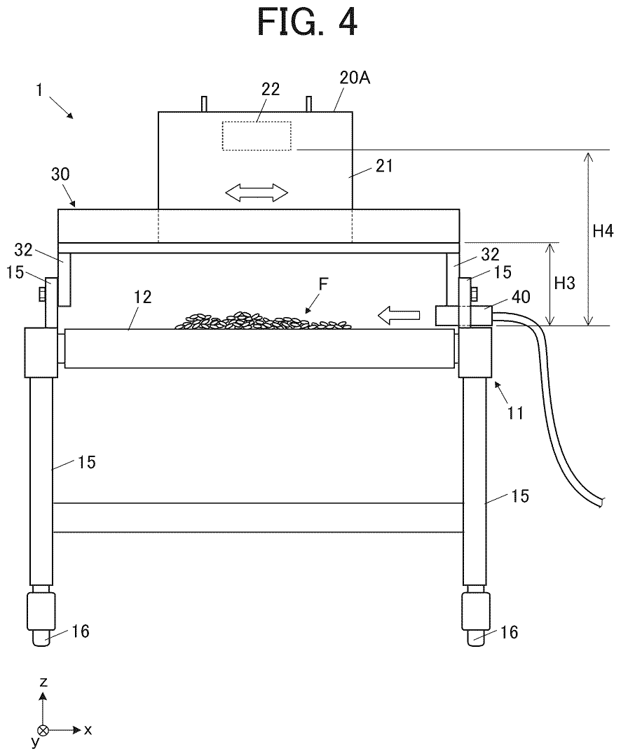

FIG. 1 is a perspective view of the schematic configuration of a food inspection apparatus 1 after an optical unit 20A is set on a conveyance mechanism 10 according to the present embodiment as an example. FIG. 2 is a perspective view of the schematic configuration of the food inspection apparatus 1 after the optical unit 20A is set on the conveyance mechanism 10 according to the present embodiment as an example. FIG. 3 is a side view of the schematic configuration of the food inspection apparatus 1 after the optical unit 20A is set on the conveyance mechanism 10 according to the present embodiment as an example. FIG. 4 is a front view of the schematic configuration of the food inspection apparatus 1 after the optical unit 20A is set on the conveyance mechanism 10 according to the present embodiment as an example.

In FIG. 1 and other figures, the width direction (left-right direction) of a belt 12 of the conveyance mechanism 10, which are described later, is defined as the x-axis direction; and the extending direction (front-rear direction) of the belt 12 is defined as the y-axis direction. The up-down direction perpendicular to the x-axis direction and the y-axis direction of the belt 12 is defined as the z-axis direction. The extending direction of the belt 12 is the conveyance direction D1 of the belt 12. In FIG. 3 and FIG. 4, the processing apparatus main body 50 is omitted for convenience.

The food inspection apparatus 1 is installed in a manufacturing line for manufacturing food F. The food inspection apparatus 1 performs quality inspection of the food F by imaging the conveyed food F and acquiring inspection information on the food F. Examples of the food F include cooked rice coated with a coating agent for frozen food, such as oil. The inspection information includes, for example, spectroscopic data on moisture absorption of the food F. As illustrated in FIG. 1, the food inspection apparatus 1 includes the conveyance mechanism 10, the optical unit 20A, a stand 30, a cooling mechanism 40, and the processing apparatus main body 50.

The conveyance mechanism 10 includes a drive section 11, an endless belt 12, and support members 13a and 13b. The drive section 11 moves the belt 12 along the conveyance direction D1. For example, the drive section 11 may be constituted of rollers, a motor, and so forth; and the motor may be driven to rotate the rollers to move the belt 12 in close contact with the rollers. For another example, the drive section 11 may be constituted of a chain, a gear, a motor, and the like; and the motor may be driven to rotate the gear engaging with the chain to move the belt 12.

The belt 12 is stretched around the rollers disposed at a predetermined interval and is moved in the conveyance direction DI by the drive section 11. The food F is placed on the top surface of the belt 12 so as to spread in a planar shape. As the belt 12 moves in the conveyance direction D1, the food F on the top surface of the belt 12 passes through an imaging area where the imager 22 performs imaging.

The support members 13a and 13b support the drive section 11 and the belt 12. The support members 13a and 13b are disposed on the left and right sides of the belt 12 and extend in the conveyance direction D1 of the belt 12. Although not illustrated, the conveyance mechanism 10 may include rails and rollers that directly support the belt 12 in addition to the support members 13a and 13b. A leg 15 that supports the belt 12 and so forth is attached to the support member 13b. The support member 13b extends downward. To the lower ends of the support member 13b, casters 16 as an example of a transportation mechanism are attached. Thus, the conveyance mechanism 10 can be moved freely, and the position of the conveyance mechanism 10 in the manufacturing line can be changed easily. The conveyance mechanism 10 may be stationary.

To the upper top surfaces of the support members 13a, one ends of attaching brackets 14 are attached by fastening members, such as screws. The attaching brackets 14 are for attaching the stand 30. To the other ends of the attaching brackets 14, legs 32 of the stand 30 (described later) are attached by fastening members, such as screws. In the present embodiment, the attaching brackets 14 are attached to the left and right support members 13a at positions corresponding to the four legs 32 of the stand 30. The attaching brackets 14 are an example of attachment members.

The optical unit 20A is movably disposed above the belt 12. Specifically, when the optical unit 20A is placed on the stand 30, the optical unit 20A is movable in the left-right direction on the stand 30 so that the operator can manually move the optical unit 20A to the position of the conveyed food F on the top surface of the belt 12. A handle is attached to the top surface of the optical unit 20A so that the operator can carry the optical unit 20A. The position of the optical unit 20A may not be manually adjusted. For example, a sensor may detect the positional shift of the optical unit 20A with respect to the food F; and, based on the detection result by the sensor, a machine may automatically adjust the position of the optical unit 20A. The optical unit 20A images the food F on the conveyed belt 12 to acquire inspection information on the food F. The optical unit 20A outputs the acquired inspection information on the food F to an information processing apparatus 52 (described later) via a wire 26.

As shown in FIGS. 1, 3 and 4, the optical unit 20A is disposed on the stand 30. The stand 30 supports the optical unit 20A such that the optical unit 20A is movable. The stand 30 also determines the height of the imager 22 of the optical unit 20A from the top surface of the belt 12. That is, the stand 30 serves as a member that adjusts the focus of the imager 22 of the optical unit 20A on the food F placed on the top surface of the conveyed belt 12. The stand 30 includes an adjustment mechanism configured to adjust the height of the stand 30 from the top surface of the belt 12. Thus, even when the imager 22 on the stand 30 is slightly unfocused, the focus of the imager 22 can be adjusted by adjusting the height of the stand 30 with the adjustment mechanism. Although the stand 30 is attached to the conveyance mechanism 10 in FIG. 1 as an example, the stand 30 may be attached to the optical unit 20A.

As shown in FIGS. 1, 3, and 4, the cooling mechanism 40 cools the food F, the stand 30, and the components of the optical unit 20A in the surrounding area by blowing air to the conveyed food F on the top surface of the belt 12 and the stand 30. The cooling mechanism 40 includes a blowing section 41 and an air supply section 42. The blowing section 41 is disposed on one end of the belt 12 in the width direction and in a space between the belt 12 and the stand 30. Specifically, the blowing section 41 is an air nozzle. The blowing section 41 is disposed such that its blowing port faces the food F on the top surface of the belt 12 or the stand 30. The blowing section 41 blows air in the direction of the arrow D2. The air supply section 42 is, for example, an air compressor. The air supply section 42 is connected to the blowing section 40 via an air tube 43. By driving the air supply section 42, air is supplied to the blowing section 41 via the air tube 43.

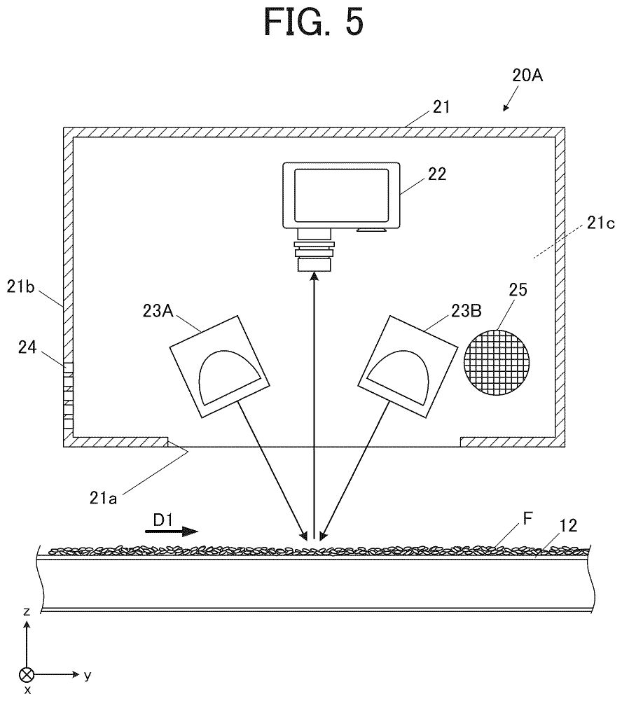

Although the blowing section 41 is an air nozzle in FIG. 1 as an example, the present invention is not limited thereto. For example, the blowing section 41 may be a fan. Although only one blowing section 41 is provided on one end of the belt 12 in the width direction, the blowing section 41 may be provided on the other end of the belt 12 in the width direction or on both ends of the belt 12 in the width direction. In such a case, multiple blowing sections 41 may be provided on one end and/or the other end of the belt 12 in the width direction. Further, multiple air outlets or multiple blowing sections 41 may be provided to blow air to the food F, the window member 33 of the stand 30, and the optical unit 20A, respectively. Further, the air blowing directions may be toward the window member 33 of the stand 30 and the edge of the housing 21 of the optical unit 20A (see FIG. 5) to prevent condensation on the window member 33 and the housing 21, for example.

The processing apparatus main body 50 is disposed on the floor around the conveyance mechanism 10. The processing apparatus main body 50 houses the information processing apparatus 52, which is heavy. The processing apparatus main body 50 includes, for example, a housing 51 constituted pf two sections: a lower section 51a and an upper section 51b. The lower section 51a of the housing 51 is, for example, a drawer, and a handle is attached to the front of the housing 51. The lower section 51a of the housing 51 houses the information processing apparatus 52, a power tap 55, and so forth. On the four positions of the bottom surface of the housing 51, casters 56 are attached as an example of a transportation mechanism. Thus, the processing apparatus main body 50 can be moved freely, and the position of the processing apparatus main body 50 in the manufacturing line can be changed easily. The information processing apparatus 52 may be mounted on the optical unit 20A, depending on the weight of the information processing apparatus 52.

The information processing apparatus 52 is, for example, a computer. The information processing apparatus 52 includes a processor that performs control and calculations, such as a CPU, and a memory, such as a RAM. CPU is an abbreviation for Central Processing Unit. RAM is an abbreviation for Random Access Memory. The information processing apparatus 52 is connected to the conveyance mechanism 10 and the optical unit 20A via the wires 26 and controls the conveyance mechanism 10 and the optical unit 20A. For example, when a multispectral camera is used as the imager 22, the processing apparatus 52 acquires spectral data of multiple wavelengths (inspection information) on the moisture absorption of the food F from a multispectral image of the food F obtained by the imager 22. The processing apparatus 52 analyzes the obtained spectral data of the multispectral image of the food F to calculate information indicating a two-dimensional distribution of the moisture content (amount of water) of the food F. As the information indicating the two-dimensional distribution of the moisture content of the food F, the processing apparatus 52 creates a two-dimensional distribution image. The two-dimensional distribution image is a heat map separately colored with multiple tone values or multiple colors.

The display part 35 is, for example, a liquid crystal display or an organic EL display. The EL is an abbreviation for Electro Luminescence. The display part 35 is attached to the side surface of the housing 51 via an arm, for example. The display part 35 is arranged near the conveyance mechanism 10. Thus, the operator can quickly check the moisture content of the conveyed food F on the top surface of the belt 12. For example, the display part 35 displays the two-dimensional distribution image and the statistical values of the moisture content in a predetermined region of the two-dimensional distribution image on the screen, based on the display data output by the processing apparatus 52. Although the display part 35 is attached to the processing apparatus main body 50 in the present embodiment, the display part 35 may be provided on a stand or may be installed in a room different from the room of the manufacturing line.

A calibration member 53 is disposed on the bottom surface of the upper section 51b of the housing 51. Specifically, the calibration member 53 is disposed at a position where the distance between the imager 22 of the optical unit 20A and the top surface of the belt 12 of the moving mechanism 10 can be recreated. The calibration member 53 is used to adjust and calibrate the light reflectivity and the like of the image information obtained by the imager 22 of the optical unit 20A. As the calibration member 53, a standard diffusion plate is used, for example. In the top surface of the upper section 51b of the housing 51, an opening 54 is formed so that the imager 22 can image the calibration member 53. The size of the opening 54 is determined to prevent fall of the optical unit 20A. The opening 54 has an edge to support the optical unit 20A. The top of the upper section 51b may not be the opening 54, but a window member made of a transparent material may be attached to the top of the upper section 51b. The processing apparatus main body 50 may include an adjustment mechanism configured to adjust the height of the bottom surface on which the calibration member 53 is disposed and/or adjust the height of the window member on which the optical unit 20A is placed.

### Internal Configuration Example of Optical Unit 20A

FIG. 5 is a diagram illustrating an example of the internal configuration of the optical unit 20A according to the present embodiment. The optical unit 20A includes a housing 21, the imager 22, light sources 23A and 23B, intake slits 24, and an exhaust port 25. In FIG. 5, the optical unit 20A is set on the conveyance mechanism 10 such that the light sources 23A and 23B are aligned in the conveyance direction D1 of the belt 12 as an example. The orientation of the optical unit 20A with respect to the conveyance mechanism 10 is not limited to the examples of FIG. 5 and other figures.

The housing 21 has a box shape and houses the imager 22 and the light sources 23A and 23B. The housing 21 is made of a metal, for example. In the bottom surface of the housing 21, an opening 21a that faces the belt 12 is formed. The opening 21a allows light to pass through, such as (i) the light including the wavelength components emitted by the light sources 23A, 23B and used in the inspection and (ii) the reflection light reflected by the food F placed on the top surface of the belt 12.

The imager 22 is, for example, a multispectral camera. The imager 22 images the food F conveyed in the conveyance direction D1 in the imaging area by the belt 12 and obtains a multispectral image of the food F. The imager 22, which includes at least a near-infrared region in its imaging wavelength range, disperses light into multiple wavelength bands to perform imaging. The imager 22 generates a data cube constituted of superposed two-dimensional (x and y axes) plane images of the food F (imaging target) for the respective spectral wavelength regions. The multispectral camera as the imager 22 may include a hyperspectral camera. The imager 22 is not limited to the multispectral camera but may be, for example, an infrared camera.

The light sources 23A and 23B include, for example, halogen lamps and LEDs. The light sources 23A and 23B are disposed at a predetermined distance from each other in the front-rear direction with respect to the imager 22, for example. The light sources 23A and 23B emit light including wavelengths used in the inspection toward the food F in the imaging area on the top surface of the belt 12. The light emitted by the light sources 23A and 23B is reflected by the food F conveyed by the belt 12, and the reflected light is imaged by the imager 22. The food inspection apparatus 1 may include a sensor that detects a decrease in the intensity of light emitted by the light sources 23A and 23B.

The air intake slits 24 are formed, for example, in the front wall 21b of the housing 21. The air intake slits 24 allow outside air to flow into the housing 21. The air outlet 25 is provided, for example, on the downstream side of the air intake slit 24 and is formed in the side wall 21c of the housing 21. The air outlet 25 discharges the air in the housing 21 to the outside. The inside of the housing 21 can be efficiently ventilated by the air intake slits 24 and the air outlet 25. The ventilation prevents stagnation of water vapor in the housing 21 and occurrence of dew condensation.

### Configuration Example of Stand 30

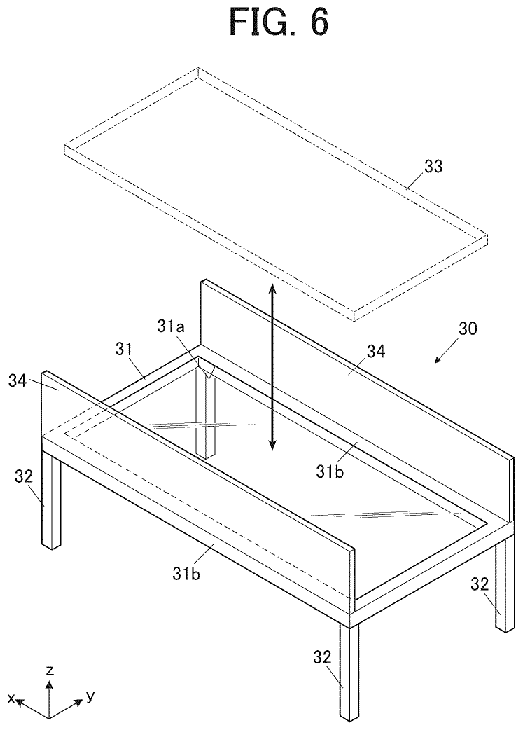

FIG. 6 is a diagram illustrating an example of the configuration of the stand 30 according to the present embodiment. The stand 30 includes a frame 31, the four legs 32, the transparent window member 33, and a pair of guide members 34. The frame 31 has a rectangular shape in a plan view. The inner edge of the frame 31 forms an opening 31a to which the window member 33 is attached. The opening 31a has a size that allows the imager 22 to image the conveyed food F. The width of the frame 31 in the short-side direction is at least equal to or longer than the width of the optical unit 20A in the y-axis direction (see FIG. 1). The width of the frame 31 in the long-side direction is at least equal to or longer than the width of the belt 12 in the x-axis direction (see FIG. 1).

The legs 32 are attached to the respective corners of the frame 31 and extend downward from the corners by a predetermined length. The legs 32 have screw holes (not-illustrated). Fastening members, such as screws, are fitted into the screw holes in the legs 32 and the screw holes of the attaching brackets 14 of the conveyance mechanism 10, thereby fastening the four legs 32 to the corresponding four attaching brackets 14. It is preferable that the length of the legs 32 be determined, based on the designed distance between the imager 22 of the optical unit 20A and the top surface of the belt 12 of the conveyance mechanism 10. Each of the legs 32 may have multiple screw holes in the height direction so that the height of the stand 30 from the top surface of the belt 12 can be adjusted stepwise. For another example, the screw holes may be elongated holes so that the height of the stand 30 from the top surface of the belt 12 can be adjusted continuously. The adjustment mechanism that adjusts the height of the legs 32 may be a jack, for example. Further, the stand 30 may not be attached to the conveyance mechanism 10 with the fastening members (e,g., screws) but may be attached by any other known method.

The transparent window member 33 has a predetermined thickness and has a shape substantially equal to or slightly greater than the external shape of the opening 31a of the frame 31. The window member 33 is removably attached to the opening 31a of the frame 31. The window member 33 may be fitted into the opening 31a of the frame 31, may be mounted on the frame 31, or may be attached to the frame 31 by being inserted to the insertion hole formed in the lateral surface of the frame portion 31. Being transparent means having a high transmittance at the wavelength of light. Specifically, the window member 33 is made of a material that can transmit the light emitted by the light sources 23A and 23B and that can transmit the light reflected by the conveyed food F. The material of the window member 33 is, for example, polycarbonate, and has a glass transition temperature [Tg] of 170° C. or higher. The material of the window member 33 is not limited to polycarbonate as long as the material is transparent and resistant to heat. Attachment and detachment of the window member 33 to and from the frame 31 may be automated by a machine, for example. Further, the food inspection apparatus 1 may include a sensor that detects stains and so forth of the window member 33. In this case, the information processing apparatus 52 may determine the timing of cleaning the window member 33, based on the detection result of the sensor, for example. Further, the window member 33 may not be detachable but may be fixed to the frame 31.

The guide members 34 are elongated plate members. The guide members 34 stand in the z-axis direction and face the long side members 31b that constitute the frame 31. Thus, the guide members 34, which serve as walls having a predetermined height, are provided on the frame 31 in the direction perpendicular to the conveyance direction D1 (the direction intersecting the conveyance direction D1). It is preferable that the distance between the guide members 34 in the y-axis direction be slightly greater than the width of the optical unit 20A in the y-axis direction. Thus, when the optical unit 20A on the stand 30 is moved in the left-right direction, the movement of the optical unit 20A in the front-rear direction is limited by the guide members 34. Therefore, the optical unit 20A can be moved to a position above the food F without being dropped off the stand 30.

The optical unit 20A may have a not-illustrated stopper that fixes the optical unit 20A to the stand 30, for example. For example, the stopper may be constituted of: a slider that holds the guide members 34 and slidably engages with the guide members 34; and bolts that fix the position of the slider to the guide members 34. The optical unit 20A is attached to the inner side of the slider. In such a case, after the optical unit 20A is moved to the position above the food F, the bolts are tightened from the outside to fasten the guide members 34 to the slider. Thus, the optical unit 20A attached to the slider can be fixed to a predetermined position of the stand 30.

### Example of how to set Optical Unit 20A

Next, setting of the optical unit 20A to the conveyance mechanism 10 will be described. As illustrated in FIGS. 1 to 4, the optical unit 20A is inserted between the guide members 34 of the stand 30 from one end in the x-axis direction of the stand 30 that is permanently attached to the conveyance mechanism 10, for example. The optical unit 20A may be brought close to the stand 30 and inserted between the guide members 34 from above the stand 30. The optical unit 20A is then slid on the window member 33 of the stand 30 toward the other end of the stand 30 and is stopped above a position where the food F on the belt 12 is expected to pass through. In an actual inspection, when the position of the optical unit 20A does not match the position of the food F on the top surface of the belt 12, the position of the optical unit 20A can be adjusted by moving the optical unit 20A along the guide members 34.

### Example of Calibrating Optical Unit 20A

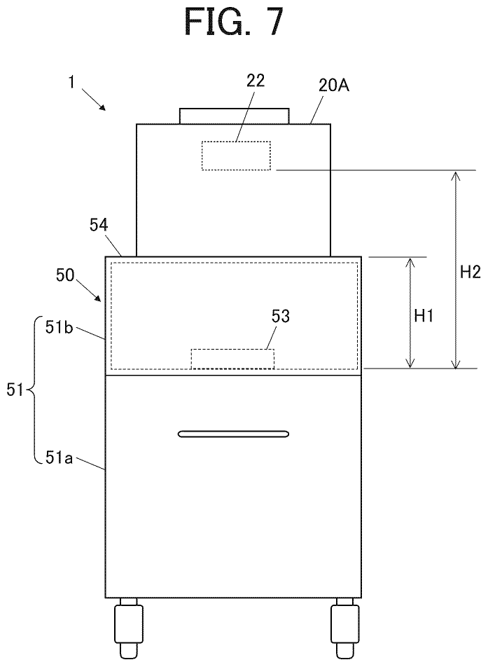

FIG. 7 is a diagram for explaining how reference information is obtained in the present embodiment. The reference information is used in calibrating spectral data obtained by the imager 22. The height H1 from the bottom surface to the top surface of the upper section 51b of the housing 51 is equal to the height H3 from the top surface of the belt 12 to the top surface of the window member 33 of the stand 30 in FIG. 4.

First, the optical unit 20A is placed on the top surface of the upper section 51b of the housing 51. The height H2 of the imager 22 of the optical unit 20A from the calibration member 53 in the upper section 51b of the housing 51 is substantially equal to the height H4 (see FIG. 4) of the imager 22 of the optical unit 20A from the top surface of the belt 12 when the optical unit 20A is placed on the stand 30 in an actual inspection. When the height H2 at the housing 51 is not equal to the height H4 at the conveyance mechanism 10, the height H2 at the housing 51 may be adjusted by adjusting the height of the calibration member 53 with an adjustment mechanism, for example. The adjustment mechanism for adjusting the height of the imager 22 may be provided to the optical unit 20A.

The light sources 23A and 23B of the optical unit 20A emit light that includes wavelength components used for the inspection to the calibration member 53 in the upper section 51b of the housing 51. The imager 22 acquires light reflected by the calibration member 53. The information processing apparatus 52 generates reference information on the reflection performance of the reflected light acquired by the imager 22. In an actual inspection of the moisture content of the food F, the imager 22 of the optical unit 20A acquires spectroscopic data of the food F conveyed by the belt 12. The information processing apparatus 52 calibrates the acquired spectral data, based on the reference information acquired by the processing device main body 50 in advance.

According to the present embodiment, the window member 33 is attached to the stand 30 provided between the conveyance mechanism 10 and the optical unit 20A. Therefore, steam, oil, and so forth generated by the food F conveyed by the belt 12 adhere to the window member 33 disposed below the optical unit 20A. The window member 33 thus prevents steam, oil, and so forth of the conveyed food F from directly adhering to the imager 22 of the optical unit 20A. Furthermore, in the present embodiment, the window member 33 of the stand 30 is removably attached to the frame 31. Therefore, when the window member 33 is stained with oil or the like, the window member 33 can be removed from the frame 31 and easily cleaned.

When the reflectivity of the food F (measurement target) is low in the inspection of the amount of moisture in the food F, the light sources 23A and 23B need to output high outputs. In such a case, the increase of the temperature around the light sources 23A and 23B may cause deformation and breakage of components of the stand 30, the conveyance mechanism 10, and the optical unit 20A. Since the cooling mechanism 40 including the blowing section 41 is provided in the present embodiment, the stand 30, the conveyance mechanism 10, and the optical unit 20A can be efficiently cooled. Such a configuration can prevent deformation and breakage of the components of the stand 30, the conveyance mechanism 10, and the optical unit 20A.

According to the present embodiment, the optical unit 20A is movably and detachably placed on the stand 30 that is attached to the conveyance mechanism 10. Thus, for example, in cleaning the belt 12 of the conveyance mechanism 10, the optical unit 20A can be easily removed from the conveyance mechanism 10. Further, when the heavy information processing apparatus 52 is disposed in the processing apparatus main body 50, the optical unit 20A can be made light and transported further easily. Accordingly, the conveyance mechanism 10 can be cleaned efficiently. Further, the movable optical unit 20A can be placed easily on various apparatuses.

### Modification Example

Next, a modification example of the above-described food inspection apparatus 1 will be described. In the modification example, the window member 33, which is disposed between the imager 22 of the optical unit 20B and the belt 12 of the conveyance mechanism 10, is attached to the optical unit 20B instead of the stand 30. In the following, differences between the modification example and the above embodiment are mainly described, and the components common to the above embodiment are described with the same reference numerals.

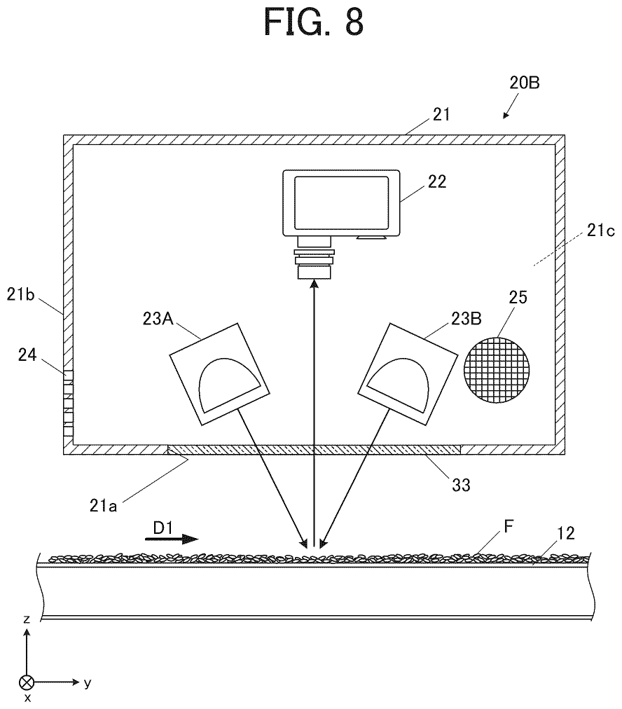

FIG. 8 is a diagram illustrating an example of the internal configuration of the optical unit 20B of the food inspection apparatus 1 according to the modification example. The optical unit 20B includes the housing 21, the imager 22, the light sources 23A and 23B, the intake slits 24, and the exhaust port 25.

The housing 21 has a box shape and houses the imager 22 and the light sources 23A, 23B. In the bottom surface of the housing 21, an opening 21a that faces the belt 12 is formed. A window member 33 is removably attached to the opening 21a of the housing 21. The window member 33 may be fitted into the 21a of the opening of the housing 21 or may be attached to the opening 21a by being inserted from the side of the housing 21. The window member 33 is made of a material that can transmit light emitted by the light sources 23A and 23B and that can transmit light reflected by the conveyed food F. The material of the window member 33 is, for example, polycarbonate, and has a glass transition temperature [Tg] of 170° C. or higher.

Since the window member 33 is attached to the optical unit 20B in the modification example, the window member 33 is not attached to the opening 31a the frame 31 of the stand 30. To secure a placement section on which the optical unit 20B is placed, it is preferable that the frame 31 be wider than the frame 31 illustrated in the figures.

According to the modification example, the window member 33 is attached to the opening 21a of the optical unit 20B. The window member 33 prevents steam, oil, and so forth of the conveyed food F from directly adhering to the imager 22 of the optical unit 20B. Furthermore, in the modification example, the window member 33 is removably attached to the frame 31 of the stand 30. Therefore, when the window member 33 is stained with oil or the like, the window member 33 can be removed from the frame 31 and easily cleaned.

Although the preferred embodiments of the present disclosure have been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art.

Although embodiments of the present invention have been described and shown in detail, the disclosed embodiments are made for purposes of illustration and example only and not limitation. The scope of the present invention should be interpreted by terms of the appended claims.

What is claimed is:

1. A food inspection apparatus configured to inspect food being conveyed by a conveyance mechanism, the food inspection apparatus comprising:
an optical unit that is disposed above the conveyance mechanism and that includes an imager, the imager being configured to image the conveyed food to acquire inspection information on the food; and
a transparent window member disposed between the conveyance mechanism and the optical unit.

2. The food inspection apparatus according to claim 1, further comprising a cooling mechanism configured to cool at least one of the food being conveyed and the window member.

3. The food inspection apparatus according to claim 1, wherein the window member is made of polycarbonate and has a glass transition temperature [Tg] of 170° C. or higher.

4. The food inspection apparatus according to claim 1, further comprising a stand on which the optical unit is placed, wherein the stand determines a height of the optical unit from the conveyance mechanism and supports the optical unit such that the optical unit is movable.

5. The food inspection apparatus according to claim 4, wherein the stand includes an adjustment mechanism that adjusts the height of the optical unit from the conveyance mechanism.

6. The food inspection apparatus according to claim 4, wherein:
the stand includes a frame having an opening, the opening allowing the imager to image the food being conveyed, and
the window member is detachably attached to the frame of the stand.

7. The food inspection apparatus according to claim 4, wherein:
the conveyance mechanism includes a support member that supports a belt on which the food is placed, and
the stand is attached to the support member of the conveyance mechanism by an attachment member.

8. The food inspection apparatus according to claim 7, wherein:
the optical unit includes a housing that houses the imager,
the housing has an opening in a surface facing the conveyance mechanism, the opening allowing the imager to image the food being conveyed, and
the window member is detachably attached to the opening.

9. The food inspection apparatus according to claim 6, wherein the stand includes a guide member that stands on the frame so as to intersect a conveyance direction of the conveyance mechanism and that guides the optical unit to a position above the food.

10. The food inspection apparatus according to claim 1, further comprising a processing apparatus main body that houses an information processing apparatus, the information processing apparatus being connected to the optical unit and configured to analyze the inspection information obtained by the imager of the optical unit.

11. The food inspection apparatus according to claim 10, wherein at least one of the processing apparatus main body and the conveyance mechanism is portable.

12. The food inspection apparatus according to claim 10, wherein:
the processing apparatus main body includes a housing, and
a calibration member is positioned in the housing such that a distance between the imager of the optical unit and a top surface of a belt of the conveyance mechanism is recreated when the optical unit is placed on the housing.
